(19) United States
(12) Patent Application Publication
(10) Pub. No.: US 2021/0196950 A1
(43) Pub. Date: Jul. 1, 2021

(54) DEVICE FOR PAIN TREATMENT

(71) Applicant: ELOSAN AG, Grabs (CH)

(72) Inventor: Alois BISCHOF, Gams (CH)

(73) Assignee: ELOSAN AG, Grabs (CH)

(21) Appl. No.: 17/199,713

(22) Filed: Mar. 12, 2021

Related U.S. Application Data
(63) Continuation of application No. PCT/EP2019/073305, filed on Sep. 2, 2019.

(30) Foreign Application Priority Data
Sep. 20, 2018 (CH) 01136/18

(51) Int. Cl. A61N 1/20; A61N 1/10; A61N 1/08

(52) U.S. Cl. CPC A61N 1/205; A61N 1/08; A61N 1/10

(57) ABSTRACT

The present disclosure relates to a device for treating pain, including a closed, electrically insulated booth having a door and a DC voltage generator for high voltages. The DC voltage generator is electrically connected to an electrode that projects inside the booth and can be grasped by a patient (P) present in the booth. The booth has a counter-electrode that runs substantially along the entire booth and that can be adjusted to a defined electric potential.

### RELATED APPLICATIONS

This application claims priority as a continuation application under 35 U.S.C. § 120 to PCT/EP2019/073305, which was filed as an International Application on Sep. 2, 2019 designating the U.S., and which claims priority to Swiss Application 01136/18 filed in Switzerland on Sep. 20, 2018. The entire contents of these applications are hereby incorporated by reference in their entireties.

### FIELD

The present disclosure relates to a device for pain treatment.

Medicinal and/or physical therapies are applied in pain treatment. The physical therapies which are applied, apart from various forms of heat and cold treatments, also include electrotherapies. Electrotherapies include for example dorsal column stimulation by way of implanted therapy apparatus which are controllable from the outside, trans-cutaneous electrical nerve stimulation, concerning which amongst other things the thin nerve fibres of the sympathetic nervous system are stimulated, galvanisation by which electrokinesis, electrophoresis and electroosmosis are utilised to bring ions and molecules in the organism into motion for pain-killing effects, iontophoresis by which electrical direct current assists in the transport of medication through the skin, treatment with Faraday alternating current by which direct current impulses are applied for a pain-reducing effect, and treatment with low-frequency, medium-frequency and high-frequency currents for stimulating nerves and muscles and for producing skin stimulations. The electrotherapies act in a predominantly local manner between two applied electrodes or between capacitors in a radiation region of an irradiator or in the field of applied magnets.

A physical therapy method, by which the complete body of a patient is included in the pain treatment, uses static electricity. Concerning this type of pain treatment, the patient is briefly subjected to high voltage. An electrical decoupling of chronically synchronised pain conductors between muscles, ligaments, nerves and the central nervous system is to be effected by way of the repeated, brief application of the high voltage. Possible blockages are to be released by way of this. The relaxation which results from this can lead to an opening of the “circulus vitiosus” between pain, tension, pain amplification resulting therefrom and a further increase in tension. An apparatus for treating the human body with static electricity, the apparatus including a closed, electrically insulated booth with a door is known. A DC voltage generator for high voltage is arranged on the booth and is electrically connected to an electrode which projects within the housing and which can be gripped in the booth by the patient. On operation, the complete surface of the body of the patient is subjected to static electrical charging, such as a negative charging, via the projecting electrode. On account of depositing electrostatic charge onto the body surface, polarisation effects occur and these act upon electrically polar molecules (e.g. water, protein chains) in the body of the patient and lead to a polarised alignment of the molecules and by way of this to a positive influencing of the colloidal states of the body fluids. It is particularly with rheumatic diseases of the soft tissue that this can lead to an alleviation of the complaints. For discharging, the patient must then step from an insulating platform onto a mat which discharges static electricity.

Effectiveness of the pain treatment with known devices is however insufficient, since the electrostatic field which is built up within the booth acts upon the body of the patient with a very non-uniform manner and results in the electrostatic charge being distributed in a very non-uniform manner on the surface of the body. Very non-uniform polarisation effects result from this, and these can lead to an insufficient physiological influencing of the colloidal states of the body fluids.

### SUMMARY

A device is disclosed for pain treatment, the device comprising: a closed, electrically insulated booth with a door; and a DC voltage generator for high voltage which is electrically connected to an electrode which projects within a housing and which is configured to be gripped by a patient (P) within the booth, wherein the booth includes a counter-electrode which extends essentially completely along the booth and which is configured to be adjustable to a defined electrical potential.

### BRIEF DESCRIPTION OF THE DRAWINGS

Further advantages and features result from the following description of exemplary embodiments of the present disclosure with reference to drawings which are not drawn to scale, and wherein:

FIG. 1 shows a schematic representation of an axially sectioned device according to an exemplary embodiment of the present disclosure, with a patient and indicated field lines of an electrical field; and

FIG. 2 schematically shows an exemplary opened device for pain treatment.

### DETAILED DESCRIPTION

A device for pain treatment is disclosed, the device permitting the electrostatic charging which can be transmitted via an electrode onto the body of the patient, to be distributed on the body surface in a uniform as possible manner.

The present disclosure puts forward a device for pain treatment, the device including a closed electrically insulated booth with a door, and a DC voltage generator for high voltage. The DC voltage generator is electrically connected to an electrode which projects within the booth and which can be gripped by a patient. The booth includes a counter-electrode which extends essentially along the complete booth and can be adjusted to a defined electrical potential.

In contrast to a booth which is known and by which the electrostatic field which acts upon the patient represents no clearly defined variable, the booth according to the present disclosure can include a counter-electrode which can be adjusted to a defined electrical potential. By way of this, the electrostatic field which acts upon the patient in the booth is clearly defined and optimisable. This results in a more uniform distribution of the electrostatic charge over the body surface of the patent, which also leads to very uniform polarisation effects. The electrostatic field which acts upon the patient is no longer dependent on any potentials which randomly arise in the booth and which can be very greatly influenced by wall distances and/or earth potentials which are randomly present in the proximity, such as e.g. metallic fittings, leads and the like.

In contrast, the electrostatic field is clearly defined and controllable by the counter-electrode. By way of this, the colloidal states of the body fluids can be physiologically positively influenced in a much more precise manner. The temporal influence of the electrostatic field upon the patient can also be controlled by way of the counter-electrode which extends essentially along the complete booth. The patient no longer needs to step from an insulating platform onto a discharge mat, but remains standing essentially within the booth in an essentially motionless manner during the complete treatment. The charging of the patient with static electricity and the discharging of the patient are effected via the electrode which projects within the housing. By way of this, an influence of the patient upon the treatment is largely ruled out.

For practical reasons, concerning an exemplary embodiment of the device according to the present disclosure, the counter-electrode can be set to earth potential for practical reasons. The counter-electrode forms a defined counter-potential with respect to the electrostatically charged patient. By way of the counter-electrode being set to earth potential, a type of shielding is created, the shielding leading and limiting the propagation of the electrostatic field in a controlled manner. As a result of the counter-electrode which is set to earth potential, the booth forms a reverse Faraday cage which restricts the electrostatic field to the inside of the booth.

An exemplary embodiment of the present disclosure can envisage the counter-electrode being configured and designed as an electrically conductive mesh, for example as a metal mesh grid, which encompasses the delimitation walls of the booth. The electrically conductive mesh can also be embedded into the delimitation walls of the booth or also be arranged on inner walls of the delimitation walls which encompass an interior of the booth. Combinations of the outlined exemplary embodiments are also possible.

Concerning an exemplary embodiment of the present disclosure, the counter electrode is arranged on inner walls of the delimitation walls which encompass an interior of the booth and are formed by an electrically conductive coating. The electrically conductive coating can be deposited onto the inner walls of the delimitation walls of the booth for example by way of spraying or by way of rollers.

An exemplary embodiment of the present disclosure can envisage the electrically conductive coating including a high-ohmic resistance. The resistance of the conductive coating is for example 200 MΩ to 1000 MΩ. On account of the high-ohmic resistance of the electrically conductive coating, a spontaneous discharge can be prevented given a random contact of the inner wall by the patient. The high resistance encourages a slower discharging, which in the case of the inner wall being touched by the patient increases patient comfort.

Concerning an exemplary embodiment of the present disclosure, the electrically conductive coating is configured and designed transparently at least in regions. Herein, the coating can be deposited onto the inner walls of the delimitation walls in a manner such that viewing regions are cleared, for example at the head height of the patient. In such cases, the coating itself can be for example opaque. Alternatively, the coating itself can be transparent. In this case, special viewing regions on the inner walls can be done away with.

In order for the operating personnel of the booth who stand outside it not to be inadvertently subjected to the electrostatic field, concerning an exemplary embodiment of the present disclosure, the electrically conductive coating can be deposited onto the inner walls of the booth in a manner such that the electrical field is shielded and is restricted to the interior of the booth.

By way of the booth having outwardly curved delimitation walls, as with an exemplary embodiment of the present disclosure, the booth can include an organic shaping. The shaping results in the distance of a patient who stands roughly centrally within the booth, to the delimitation walls, being roughly equally large at all sides. This assists in the uniformity of the electrostatic field which acts upon the patient. For assisting the organic shaping of the booth further, this in an exemplary embodiment can include for example an outline which approximates a circular shape.

For reasons of manufacturing technology and in order to simplify the construction of the booth on location, concerning an exemplary embodiment of the present disclosure, the booth includes a number of outwardly curved segments which are connected to one another. Herein, one of the segments forms the door for the access into the inside of the booth. The booth itself has a strongly insulating floor. The dielectric strength of the floor is herein larger than for example 400 kV.

Concerning a variant of the present disclosure, the number of the segments which forms the booth is at least three. The segments are herein configured and designed predominantly of the same type. One of the segments forms the door. A second segment on its convexly curved outer side carries the DC voltage generator for high voltage which is electrically connected to the electrode which projects on the inner wall.

By way of the segments being configured and designed curved towards one another at one of their longitudinal ends, as with an exemplary embodiment of the present disclosure, the segments in the state in which they are assembled on one another close the booth to the top. On account of this, one can then make do without a separate roof for the booth.

The inner walls of the segments are provided with an electrically conductive coating which includes a high-ohmic resistance of for example preferably about 200 MΩ to about 1000 MΩ. By way of this, it can be ensured that given an inadvertent contacting of the inner wall by the charged patient, automatically a very slow, gentle discharge is effected, with regard to which only minimal currents can flow, such hardly being noticeable by the patient.

The coated inner walls of the segments are electrically connected to one another, so that the electrically conductive coating on the inner walls of all segments can be very simply set to the same electrostatic potential.

For example, glass, polycarbonate (PC), polymethyl methacrylate (PMMA), mouldable polyethylene terephthalate (PET-G) and similar preferably transparent materials can be considered as materials for the segments of the booth. Depending on the intrinsic stiffness of the segments, the booth can also yet be additionally provided with a frame which is arranged on the outer side and which supports the stability of the booth.

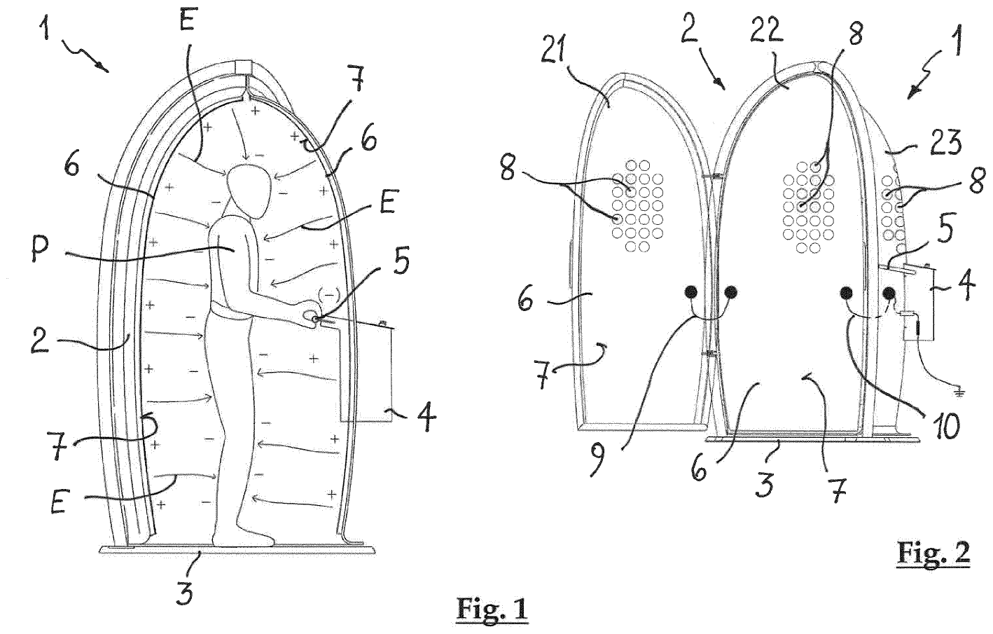

An exemplary embodiment of a device for pain treatment, represented in the axial section in FIG. 1, in its entirety has the reference numeral 1. The device 1 includes a closed, electrically insulated booth 2 which is arranged on an electrically insulating floor 3 which has a dielectric strength of for example at least 400 kV, and a direct voltage generator for generating high voltage, which together with further electrical and electronic circuit components is arranged in a housing 4 which is accessible and operable from the outside. The DC voltage generator which is arranged within the housing 4 is electrically connected to an electrode 5 which projects within the booth and which can be gripped by a patient P. For this, the electrode 5 has the shape of handle which can be gripped by the patient P with one or with both hands. The booth 2 is provided with a counter-electrode 6 which extends essentially along the entire booth and can be set to a defined electrical potential. Concerning the represented exemplary embodiment example, the counter-electrode 6 is set to earth potential.

The counter-electrode 6 can be configured and designed for example as an electrically conductive mesh, for example as a metal mesh grid which encompasses delimitation walls of the booth. The electrically conductive mesh can also be embedded into the delimitation walls of the booth or also be arranged on outer walls and/or on inner walls of the delimitation walls which encompass the interior of the booth. Combinations of the outlined exemplary embodiments are also possible. According to the represented exemplary embodiment, the counter electrode 6 is arranged on the inner walls of the delimitation walls which encompass the interior of the booth 2 and is formed by an electrically conductive coating 7. The electrically conductive coating 7 can be deposited onto the inner walls of the delimitation walls of the booth 2 for example by way of spraying or by way of rollers. The electrically conducive coating has a high-ohmic resistance which is for example at or about 200 MΩ to at or about 1000 MΩ. Given a coincidental contact of the inner wall by the patient P, a spontaneous discharge can be prevented by way of the high-ohmic resistance of the electrically conductive coating. The high resistance encourages a slower discharge, which in the case of a contact of the inner wall by the patient P increases patient comfort.

As is indicated in FIG. 1, the patient P is electrostatically charged by way of the electrical charge which is generated by the high-voltage generator and which is led into the inside of the booth 2 via the electrode 5 which is configured and designed as a handle. The electrostatic charge, in the represented exemplary embodiment a charging with negative charge carriers, propagates over the entire surface of the patient P. The electrically conductive coating 7 which is kept at earth potential, with respect to the patient P who is subjected to the negative surface charging forms a positively charged counter-electrode 6. The field lines of the electrostatic field which result in the booth 2 are indicated in FIG. 1 as arrows from the electrically conductive coating 7 to the body surface of the patient. It is to be understood that the polarity of the electrostatic field can also be the other way around, i.e. that the patient is subjected to positive charge carriers. In this case, the field lines run from the positively charged body surface of the patient to the more negative, electrically conductive coating which is turn can be set to earth potential.

The booth 2 includes delimitation walls which are outwardly curved. From this, an organic shaping of the booth 2 results, in which the distance of the patient P who is present in the booth 2 as centrally as possible, to the delimitation walls which are provided with the electrically conductive coating, is approximately uniform. By way of this, the electrostatic field which acts upon the patient is relatively uniform over the whole body surface. For a further improvement of the uniformity of the acting electrostatic field, the booth 2 can yet also have an outline which approximates a circular shape.

The discharge of the patient P is effected by way of the electrode 5 which is gripped by the patient being set to earth potential. The switch-over from electrostatic charging of the patient to patient discharging can be effected by hand. For example, the switch-over is effected via an electronic control which can likewise be arranged in the housing 4. The electrostatic charging, the time duration of the action of the electrostatic field, the discharging of the patient P, as well as the number of charging/discharging cycles can be controlled via the electronic control.

FIG. 2 schematically shows a booth 2 of a device for pain treatment 1 in the opened state. The booth 2 which is arranged on the electrically insulating floor 3 is composed of several, for example three segments 21, 22, 23 which are mechanically connected to one another. Herein, the segments are configured and designed for the most part of the same type. One of the segments, in the represented exemplary embodiment the segment 21, forms the door of the booth 2. A second segment, in the represented exemplary embodiment the segment 23, on its convexly curved outer side carries the housing 4 in which the direct voltage generator for high voltage and the further electrics and electronics are accommodated. The electrode 5 which is configured and designed as a handle and which is connected to the electronics (not shown) in the housing 4 is arranged on the inner wall of the segment 23. The segments 21, 22, 23 at their upper longitudinal ends are configured and designed curved towards one another and in the state in which they are assembled on one another close off the booth 2 to the top. One can a make do without a separate roof for the booth 2.

According to the represented exemplary embodiment, the coating 7 which forms the counter-electrode 6 is deposited onto the inner walls of the segments 21, 22, 23 in a manner such that viewing regions 8 are left roughly at the head height of the patient. The viewing regions 8 with the represented exemplary embodiment are configured and designed as a number of circular openings in the electrically conductive coating 7. However, it is to be understood that the viewing regions can also have any arbitrary other shape. In an alternative exemplary embodiment of the present disclosure, the coating 7 can itself be configured and designed in an at least partly transparent or also entirely transparent manner. In this case, one can make do without special viewing regions 8 on the inner walls.

The electrically conductive coatings 7 on the inner walls of the segments 21, 22, 23 are electrically connected to one another. This is indicated by the electrical connections 9, 10. Herein, it is yet to be noted that as a result of the selected view, the electrical connection 9 between the segments 22 and 23 is not completely visible. Furthermore, it is also indicated that the electrical coating of the inner walls of the segments is connected to earth potential in a high-ohmic manner.

For example glass, polycarbonate (PC), polymethyl methacrylate (PMMA), mouldable polyethylene terephthalate (PET-G) and similar, for example preferably, transparent materials are considered as materials for the segments 21, 22, 23 of the booth 2. Depending on the intrinsic stiffness of the segments 21, 22, 23, the booth 2 can also yet additionally be provided with a frame 11 which is arranged on its outer side and which supports the stability of the booth 2.

The present disclosure has been described with the example of specific exemplary embodiments. The aforementioned description however merely serves for the explanation of the present disclosure and is not to be considered as limiting. In contrast, the present disclosure is defined by the patent claims and the equivalents which are derived by the person skilled in the art and encompassed by the general inventive concept.

It will be appreciated by those skilled in the art that the present disclosure can be embodied in other specific forms without departing from the spirit or essential characteristics thereof. The presently disclosed exemplary embodiments are therefore considered in all respects to be illustrative and not restricted. The scope of the present disclosure is indicated by the appended claims rather than the foregoing description and all changes that come within the meaning and range and equivalence thereof are intended to be embraced therein.

1. A device for pain treatment, the device comprising:
a closed, electrically insulated booth with a door; and
a DC voltage generator for high voltage which is electrically connected to an electrode which projects within a housing and which is configured to be gripped by a patient (P) within the booth, wherein the booth includes a counter-electrode which extends essentially completely along the booth and which is configured to be adjustable to a defined electrical potential.

2. The device according to claim 1, wherein the counter electrode is configured to be set to earth potential.

3. The device according to claim 2, wherein the counter-electrode is configured and designed as an electrically conductive mesh which encompasses delimitation walls of the booth, or is embedded into delimitation walls of the booth, or is arranged on outer and/or inner walls of the delimitation walls which encompass an interior of the booth.

4. The device according to claim 3, wherein the counter electrode is arranged on inner walls of the delimitation walls which encompass an interior of the booth and is formed by an electrically conductive coating.

5. The device according to claim 4, wherein the electrically conductive coating comprises:
a high-ohm resistance which is 200 MΩ to 1000 MΩ.

6. The device according to claim 4, wherein the electrically conductive coating at least in regions is configured and designed to be transparent.

7. The device according to claim 4, wherein the electrically conductive coating is deposited onto the inner walls of the booth in a manner such that the electrical field is shielded and limited to an inside of the booth.

8. The device according to claim 7, the booth comprises:
outwardly curved delimitation walls.

9. The device according to claim 8, wherein the booth comprises:
an outline which approximates a circular shape.

10. The device according to claim 9, wherein the booth comprises:
a number of outwardly curved segments of electrically insulating material which are connected to one another, wherein one of the segments forms the door.

11. The device according to claim 10, wherein the number of segments is at least three.

12. The device according to claim 10, wherein the segments are configured and designed to be curved towards one another at their longitudinal ends and in a state in which they are assembled on one another to close the booth at a top thereof.

13. The device according to one of the claim 10, wherein the segments comprise:
inner walls which are provided with an electrically conducive coating which has a high-ohm resistance of about 200 MΩ to about 1000 MΩ.

14. The device according to claim 13, wherein the inner walls of the segments which are provided with the electrically conducive coating are electrically conductively connected to one another.

15. The device according to claim 14, wherein the segments consist of: glass, polycarbonate (PC), polymethyl methacrylate (PMMA), mouldable polyethylene terephthalate (PET-G) and/or similar transparent materials.

16. The device according to claim 1, wherein the counter-electrode is configured and designed as an electrically conductive mesh which encompasses delimitation walls of the booth, or is embedded into delimitation walls of the booth, or is arranged on outer and/or inner walls of the delimitation walls which encompass an interior of the booth.

17. The device according to claim 1, the booth comprises:
outwardly curved delimitation walls.

18. The device according to claim 1, wherein the booth comprises:
an outline which approximates a circular shape.

19. The device according to claim 1, wherein the booth comprises:
a number of outwardly curved segments of electrically insulating material which are connected to one another, wherein one of the segments forms the door.

20. The device according to claim 10, wherein the segments consist of: glass, polycarbonate (PC), polymethyl methacrylate (PMMA), mouldable polyethylene terephthalate (PET-G) and/or similar transparent materials.
